(19) United States
(12) Patent Application Publication
(10) Pub. No.: US 2024/0321434 A1
(43) Pub. Date: Sep. 26, 2024

(54) INFORMATION PROCESSING SYSTEM, MEDICAL SYSTEM, AND INFORMATION PROCESSING METHOD

(71) Applicant: OLYMPUS CORPORATION, Tokyo (JP)

(72) Inventors: Ryohei OGAWA, Tokyo (JP); Hidetoshi NISHIMURA, Tokyo (JP); Genri INAGAKI, Tokyo (JP); Mariko OTAGIRI, Sagamihara-shi (JP); Yuji SAKAMOTO, Tokyo (JP); Nao INOUE, Isehara-shi (JP); Shohei HEMMI, Tokyo (JP)

(73) Assignee: OLYMPUS CORPORATION, Tokyo (JP)

(21) Appl. No.: 18/612,092

(22) Filed: Mar. 21, 2024

Related U.S. Application Data
(60) Provisional application No. 63/454,087, filed on Mar. 23, 2023.

(51) Int. Cl. G16H 30/40; G06T 3/60; G06T 5/50

(52) U.S. Cl. CPC G16H 30/40; G06T 3/60; G06T 5/50; G06T 2207/10068; G06T 2207/10132; G06T 2207/20221

(57) ABSTRACT

An information processing system includes a processor including hardware. The processor acquires an endoscope image from an endoscope in which a papillary portion appears in the endoscope image, corrects the endoscope image based on correction information which is identified by an organ structure, and determines route information of a lumen based on the corrected endoscope image.

### CROSS REFERENCE TO RELATED APPLICATIONS

This application is based upon and claims the benefit of priority to U.S. Provisional Patent Application No. 63/454,087 filed on Mar. 23, 2023, the entire contents of which are incorporated herein by reference.

### BACKGROUND

Techniques for treating cholangiocarcinoma and the like using an endoscope have been known. U.S. Patent Application Publication No. 2017/0086929 discloses a technique regarding a remote robotic surgery system in which a treatment tool, such as a catheter, is inserted into the biliary duct through a treatment tool channel of an endoscope.

### SUMMARY

In accordance with one of some aspect, there is provided an information processing system comprising a processor including hardware, the processor being configured to: acquire an endoscope image from an endoscope in which a papillary portion appears in the endoscope image, from an endoscope; and correct the endoscope image based on correction information which is identified by an organ structure and determine route information of a lumen based on the corrected endoscope image.

In accordance with one of some aspect, there is provided a medical system comprising:
the information processing system as defined in the above; and the endoscope.

In accordance with one of some aspect, there is provided an information processing method, comprising the steps of: acquiring an endoscope image from an endoscope in which a papillary portion appears in the endoscope image, from an endoscope; and correcting the endoscope image based on correction information which is identified by an organ structure and determining route information of a lumen based on the corrected endoscope image.

### BRIEF DESCRIPTION OF THE DRAWINGS

FIG. 1 shows organs and tissues involved in ERCP procedure.

FIG. 2 is an explanatory view of a flow of ERCP procedure.

FIG. 3 is a schematic diagram of the form of a papillary portion viewed directly from the front.

FIG. 4 is an explanatory view of an example of a modified organ structure.

FIG. 5 is an explanatory view of another example of a modified organ structure.

FIG. 6 is an explanatory view of another example of a modified organ structure.

FIG. 7 is a block diagram explaining a configuration example of an information processing system.

FIG. 8 is a flowchart explaining a process example of the present embodiment.

FIG. 9 is an explanatory view of an example of a positional relationship between an imaging section of an endoscope and a papillary portion.

FIG. 10 is an explanatory view of an example of a flow of changes in image by processing of the present embodiment.

FIG. 11 shows endoscope images of papillary portions, and the corresponding classification types for the biliary duct and the pancreatic duct.

FIG. 12 is an explanatory view of processing using a trained model.

FIG. 13 is a flowchart explaining another process example of the present embodiment.

FIG. 14 is an explanatory view of another example of a positional relationship between an imaging section of an endoscope and a papillary portion.

FIG. 15 is an explanatory view of another example of a flow of changes in image by processing of the present embodiment.

FIG. 16 is a flowchart explaining another process example of the present embodiment.

FIG. 17 is an explanatory view of another example of a flow of changes in image by processing of the present embodiment.

FIG. 18 is an explanatory view of another example of a flow of changes in image by processing of the present embodiment.

FIG. 19 is an explanatory view of an example of navigation in a display device of the present embodiment.

FIG. 20 is an explanatory view of an example of further applying MRCP images to the method of the present embodiment.

FIG. 21 is an explanatory view of a configuration example of a medical system.

FIG. 22 shows the vicinity of a distal end of an endoscope in a positioning step.

FIG. 23 is a schematic view of an endoscope including a bending section and a drive mechanism thereof.

FIG. 24 is an explanatory view of a configuration example of a forward/backward drive device.

FIG. 25 is an explanatory view of a connecting section including a rolling drive device.

### DETAILED DESCRIPTION

The following disclosure provides many different embodiments, or examples, for implementing different features of the provided subject matter. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

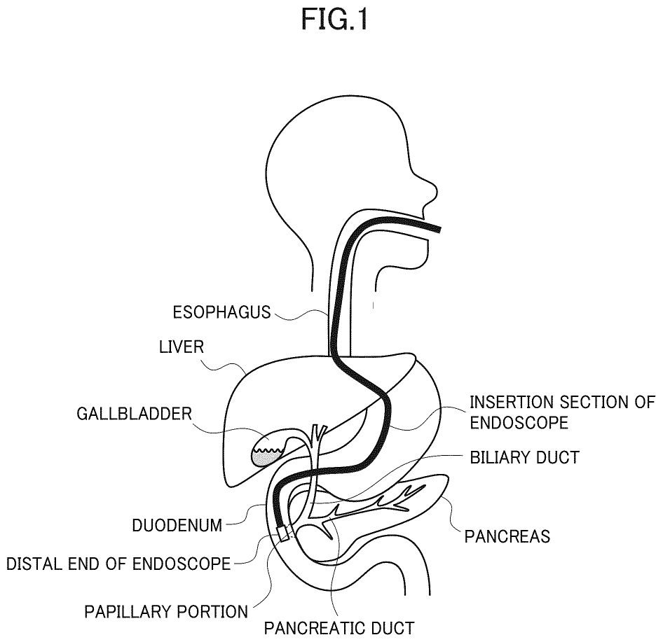

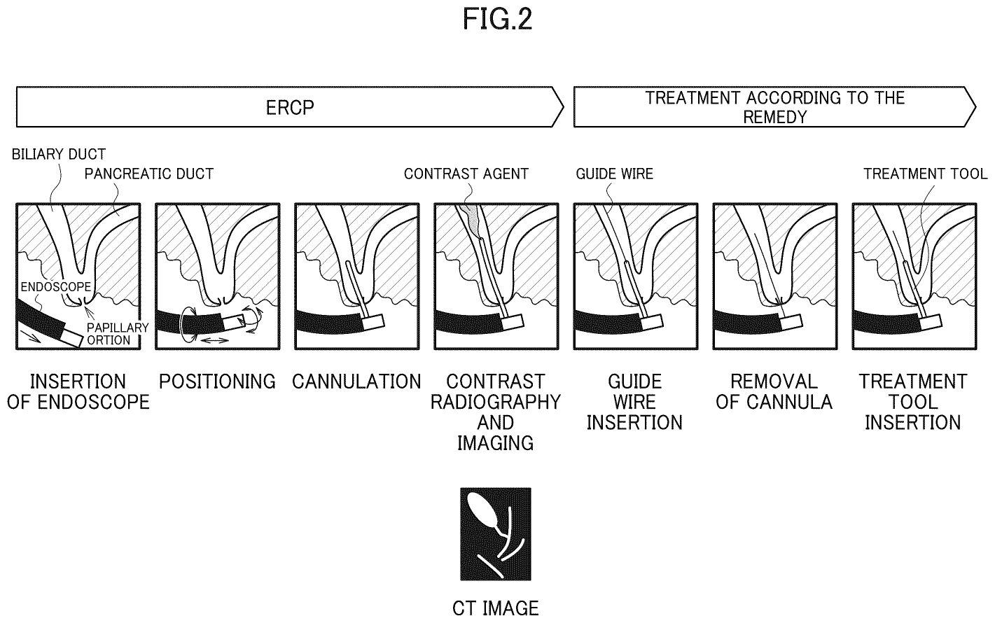

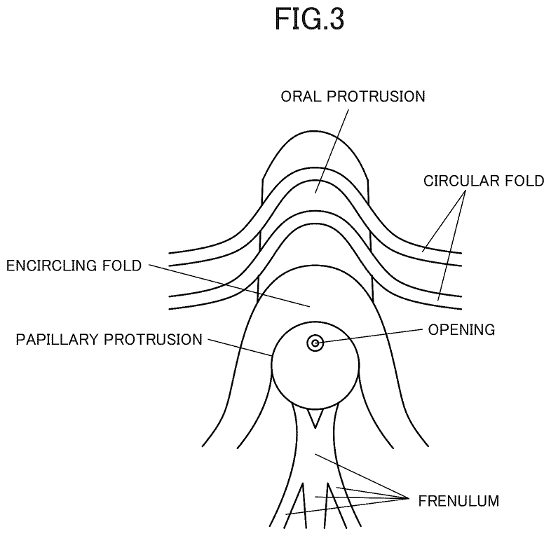

The method of the present embodiment relates to a route guide for the biliary duct and the pancreatic duct when conducting ERCP, or the like. ERCP stands for Endoscopic Retrograde Cholangio Pancreatography. The contents of the ERCP procedure are described with reference to FIG. 1, FIG. 2, and FIG. 3.

FIG. 1 shows organs and tissues involved in the ERCP procedure. The organs include a multiple types of tissues, forming a unique structure with a specific function. For example, in FIG. 1, liver, gallbladder, pancreas, esophagus, stomach, and duodenum are shown as organs. Tissues are formed by related cells combined, and examples include blood vessels, muscles, skin, and the like. For example, in FIG. 1, a biliary duct and a pancreatic duct are shown as tissues.

The example of organ structure shown in FIG. 1 is a general organ structure example that has not been modified by surgical or other treatments, which are described later. The organ structure may also be referred to as an anatomical structure of digestive tract. The digestive tract designates the stomach and the intestinal tract. If a gastrectomy has been performed, the stomach herein includes a remnant stomach section. Further, the intestinal tract includes the small intestine, the large intestine, and the like. Further, the small intestine includes the duodenum, the jejunum, and the ileum. In the case of the organ structure shown in FIG. 1, the distal end section of the endoscope is inserted from the stomach side toward the duodenum during the ERCP procedure.

The biliary duct is the target of the ERCP procedure. The biliary duct is a pipeline for allowing the bile produced in the liver to flow into the duodenum. When approaching the biliary duct using an endoscope, a treatment tool inserted into the channel of the endoscope is inserted to the biliary duct from the papillary portion of the duodenum while holding the endoscope at the position of the duodenum. Hereinafter, the papillary portion of the duodenum is simply referred to as a papillary portion. The papillary portion is a region including an opening of the luminal tissue with respect to the duodenum. Not only the opening but also the structure around the opening is referred to as a papillary portion. The opening of the luminal tissue is the opening of a common duct with respect to the duodenum. The common duct is formed as the confluence of the biliary duct and pancreatic duct. However, as described later, the papillary portion largely varies between individuals. For example, in some cases, the biliary duct opens directly to the duodenum without being merged with the pancreatic duct. In this case, the opening of the luminal tissue is the opening of the biliary duct.

FIG. 2 shows an example of a flow of ERCP procedure. Although FIG. 2 shows, as an example, a side-view type endoscope in which an imaging section, an illumination lens, and an opening of a treatment tool channel are provided on the side of the distal end section of the endoscope, the use of other endoscopes, for example, a direct-view type endoscope and the like during the ERCP is not excluded. The imaging section herein is also referred to as a camera, and includes an imaging sensor formed of a CCD (Charge-Coupled Device), a CMOS (Complementary Metal-Oxide-Semiconductor) sensor, or the like, an optical member, and the like, and functions as an imaging device. Further, in the present embodiment, an image captured by the imaging section is referred to as an endoscope image. Further, the endoscope image may be a still image created from the video captured by the imaging section.

In an endoscope insertion step, the insertion section of the endoscope is inserted from the mouth to the duodenum through the esophagus and the stomach. At this time, the insertion section is inserted until the papillary portion becomes roughly visible in the field of view of the endoscope.

Next, in a positioning step, the position of the endoscope relative to the papillary portion is adjusted. Specifically, the position of the distal end section of the endoscope is adjusted so that the papillary portion appears within the imaging range of the imaging section of the endoscope. Further, the position of the distal end section of the endoscope is adjusted so that the imaging section of the endoscope and the papillary portion have a given positional relationship. A given positional relationship is, for example, the relationship in which the distal end of the distal end section of the endoscope is facing toward the jejunum, the imaging section of the endoscope is directly facing the papillary portion, and the papillary portion is positioned in the center of the endoscope image. The expression “directly facing the papillary portion” means that the line-of-sight direction of the imaging section is substantially perpendicular to the intestinal wall, where the papillary portion is present. The expression “the papillary portion is positioned in the center of the endoscope image” specifically means, for example, that the center of a region including the encircling fold, the oral protrusion, the frenulum, and the main papilla, which are described later, is positioned substantially in the center of the endoscope image, or that the opening of the luminal tissue, i.e., the main papilla, is positioned substantially in the center of the endoscope image.

In ERCP, the vision of the endoscope image when the imaging section of the endoscope and the papillary portion have a given positional relationship can be maintained. This allows the operator to observe the papillary portion always with the same view during the cannulation step, which is described later. This allows the operator to easily grasp the progress, condition, abnormality or the like of the procedure based on past cases, experiences, and the like. Therefore, in each case of ERCP, many endoscope images with the imaging section of the endoscope and the papillary portion in a given positional relationship are acquired. Therefore, the endoscope images with the imaging section of the endoscope and the papillary portion in a given positional relationship can be used as training data for machine learning, which is described later.

Then, in the cannulation step, a cannula is inserted from the papillary portion into the biliary duct. Specifically, the cannula is inserted into the treatment tool channel of the endoscope so that the cannula protrudes from the channel opening of the distal end section of the endoscope. The distal end of the cannula is inserted into the common duct from the opening of the common duct, and the cannula is further inserted through the confluence of the biliary duct and the pancreatic duct toward the direction of the biliary duct. Cannulation refers to insertion of a cannula into a body. A cannula is a medical tube that is inserted into a body for medical purposes. The operator can always observe the papillary portion with the same view by maintaining the view of the endoscope image in the procedure, such as cannulation after the positioning step. In this way, by always observing the papillary portion with the same view, the operator can easily grasp the progress, condition, or abnormality of the procedure based on past cases or experiences, etc.

Next, in the contrast radiography and imaging step, a contrast agent is injected into the cannula and poured into the biliary duct through the distal end of the cannula. By performing X-ray or CT imaging in this state, an X-ray image or a CT (Computed Tomography) image in which the biliary duct, the gallbladder, and the pancreatic duct appear can be obtained. The procedure that has been described so far is the ERCP procedure. After the procedure, various treatments are performed according to the results of diagnosis based on the X-ray image or the CT image. An example of the treatment is described below.

In a guide wire insertion step, a guide wire is inserted into a cannula so that the guide wire is protruded from the distal end of the cannula, and the guide wire is inserted into the biliary duct. In a cannula removing step, the cannula is removed while leaving the guide wire inside the biliary duct. As a result, only the guide wire protrudes from the distal end section of the endoscope, indwelling in the biliary duct. Next, in a treatment tool insertion step, the treatment tool is inserted into the biliary duct along the guide wire. An example of a treatment tool is a basket or stent. The basket is used with a catheter. While allowing the guide wire to pass through the catheter, the catheter is inserted into the biliary duct along the guide wire. A basket made of a plurality of metal wires is inserted into the biliary duct from the distal end of the catheter, an object to be removed, such as a gallstone, is placed in the basket and held, and the object to be removed is taken out from the biliary duct by removing the basket and the catheter in this state from the biliary duct. A stent is also used in a similar manner with a catheter and inserted into the biliary duct from the distal end of the catheter. The narrow portion of the biliary duct can be widened by inserting a stent; further, by keeping the stent therein, the narrow portion is held in a widened state by the indwelling stent.

The procedure of ERCP is performed in the manner described above. However, in the cannulation step, in terms of the operator's field of view, the operator can only observe an endoscope image showing the papillary portion from the outside. For example, as schematically shown in FIG. 3, distinctive structures are present around the opening of the papillary portion. Specifically, structures called frenulum, papillary protrusion, encircling fold, circular fold, and oral protrusion are present around the opening, which is the main papilla. However, FIG. 3 is only a typical example, and the form of the papillary portion largely varies between individuals. Examples of such individual differences include unclear main papilla, frenulum, encircling fold, or oral protrusion, and other types of form that is significantly different from the typical form shown in FIG. 3. Therefore, the operator presumes the position of the opening and the direction of the biliary duct based on past cases, experiences, or the like, while viewing the endoscope image, and attempts cannulation according to the presumption.

At this time, in order to allow the operator to more appropriately presume the direction of the biliary duct, it is desirable that the captured endoscope image be one that is easy to compare with past cases or one that the operator is familiar with. As shown in FIG. 1, since the results of the ERCP cases with a general organ structure have been accumulated, it is easy to compare the endoscope images with those of past cases. However, in the ERCP treatments dealing with the organ structures that have been modified by a predetermined surgical treatment, endoscope images from which the operator cannot easily presume the route of the biliary duct are captured.

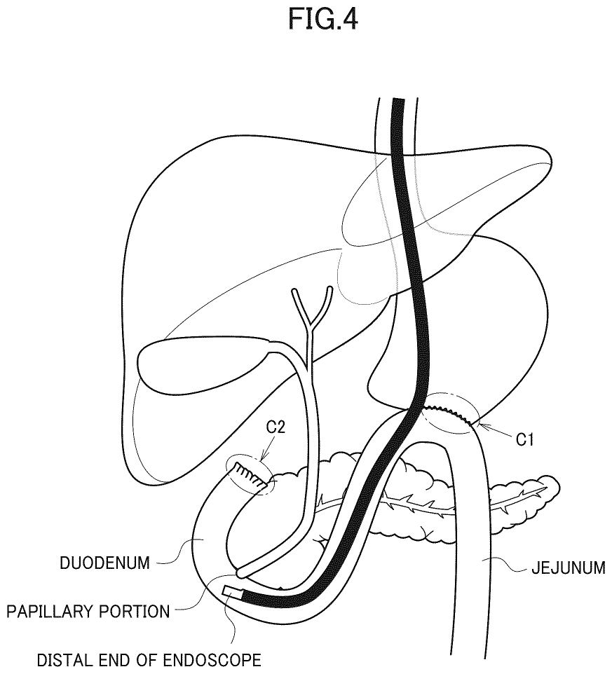

The predetermined surgical treatment is, for example, the Billroth II method. As shown in C1 in FIG. 4, in the Billroth II method, the food passage is bypassed by anastomosis of the jejunum with the stump of the remnant stomach section after resection of the pyloric side of the stomach for the purpose of obesity treatment, removal of gastric cancer, or the like. Further, as shown in C2, the end of the duodenum is closed by suture. The term “obesity” refers to a condition in which excess body fat is accumulated to the extent that, for example, it can shorten life expectancy or cause health problems. An obesity treatment surgery is performed, for example, when the BMI exceeds a certain value or when the BMI exceeds a certain value and also the patient has comorbidity. BMI stands for Body Mass Index.

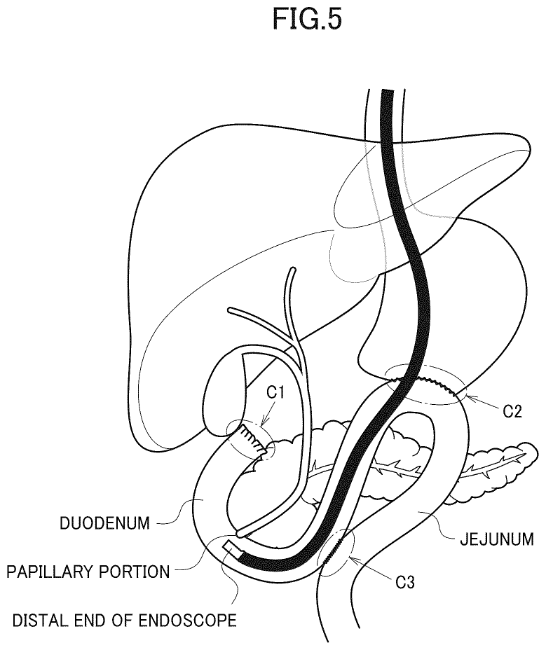

The Billroth II method herein may also include Braun anastomosis. In the Braun anastomosis, for example, the jejunum and the duodenum lifted by the Billroth II method are anastomosed as shown in C3 in FIG. 5. This prevents duodenal fluid from flowing to the end side of the duodenum closed by suture.

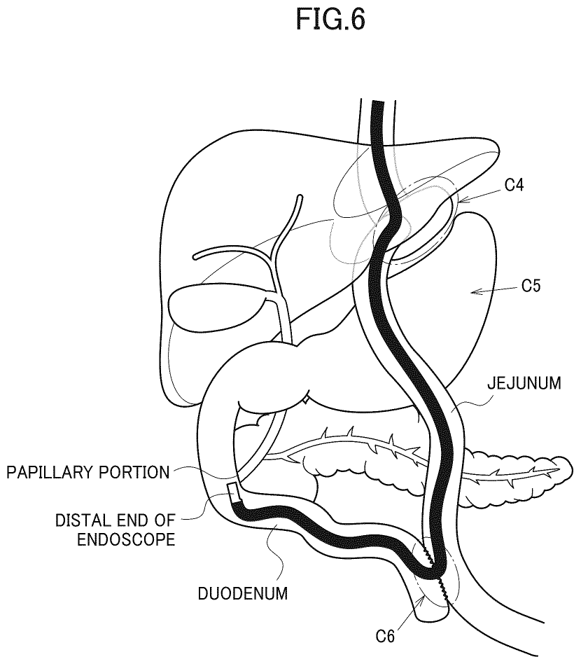

Further, the predetermined surgical treatment may be, for example, RYGB. RYGB stands for Roux-en-Y gastric bypass. The RYGB is used, for example, for an obesity treatment surgery or the like, and the stomach is divided into a small portion shown in C4 in FIG. 6 and another portion shown in C5 in FIG. 6. Then, the portion shown in C4 is anastomosed with the jejunum separated from the duodenum. Further, as shown in C6, the end of the duodenum, which is once separated, is anastomosed with the jejunum.

As shown in FIGS. 4 to 6, when the organ structure is modified, the distal end section of the endoscope is inserted from the jejunal side toward the papillary portion of the duodenum; therefore, the endoscope image captured in this case is different from the endoscope image captured in the case of FIG. 1. In other words, if the organ structure is modified, the endoscope images acquired during the ERCP treatment are not images from which the operator can easily presume the directions of the biliary duct and the pancreatic duct.

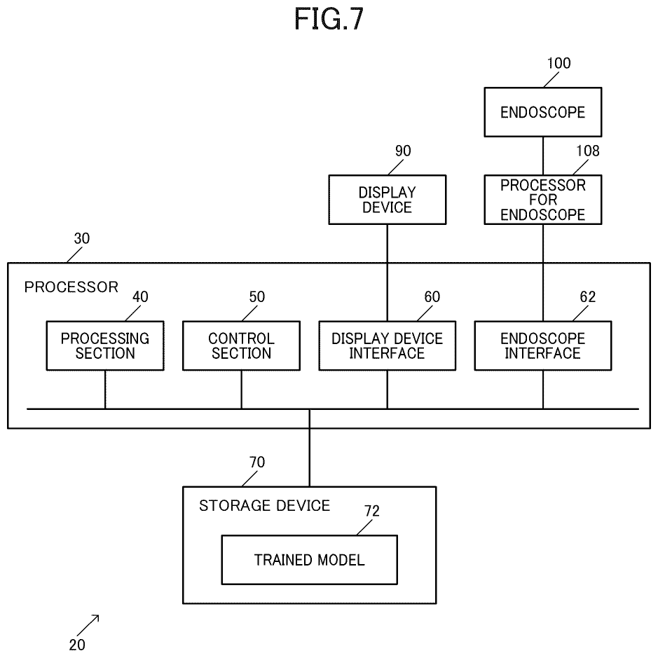

Therefore, in the present embodiment, the endoscope images are corrected based on the correction information which is identified by the organ structure, and the route information of a lumen is presumed based on the corrected endoscope images. The correction information refers to information that indicates the contents of the image correction process for correcting endoscope images. In the following explanations, “an image correction process” may also be simply referred to as “correction”. The contents of the image correction process, which are described later in detail, include the type of correction, the correction parameters, and the like. The processor 30 corrects the endoscope images according to the contents of the correction indicated by the correction information. The method of the present embodiment can be implemented by the information processing system 20 shown in the configuration example of FIG. 7.

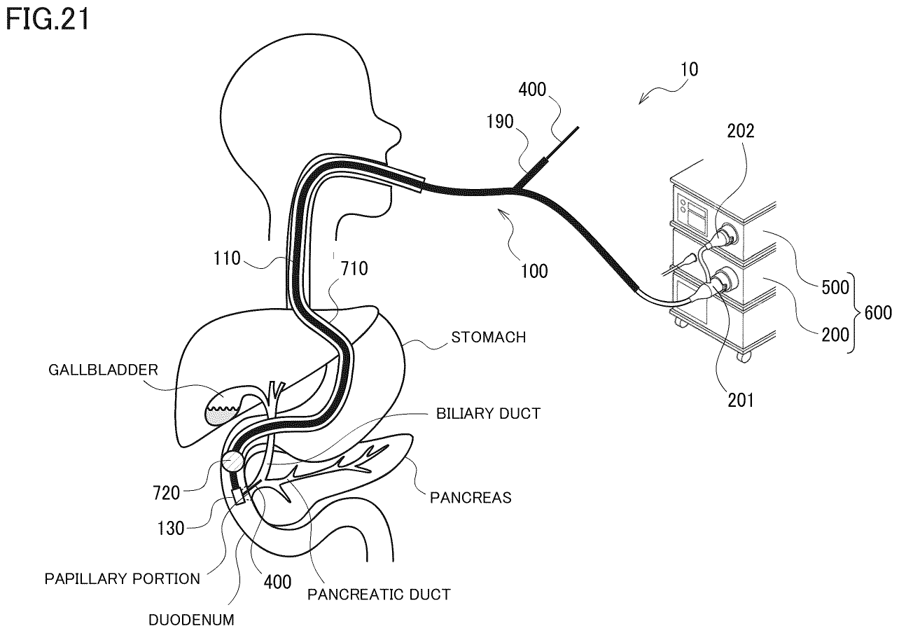

As shown in FIG. 7, the information processing system 20 includes the processor 30. The information processing system 20 may also include a storage device 70. The information processing system 20 may be implemented, for example, by a control device 600 of a medical system 10, which is described later with reference to FIG. 21. In this case, the medical system 10 is implemented by the endoscope 100 and the information processing system 20. In this case, for example, part or all of the information processing system 20 may be implemented by a drive control device 200 or a video control device 500 in the control device 600, or by an information processing device such as a PC provided in the control device 600 separately from the drive control device 200 and the video control device 500. PC stands for a Personal Computer. Alternatively, part or all of the information processing system 20 may be implemented by, for example, a server in a cloud system.

The processor 30 includes hardware. The hardware of the processor 30 may include at least one of a circuit for processing digital signals and a circuit for processing analog signals. For example, the hardware may include one or a plurality of circuit devices or one or a plurality of circuit elements mounted on a circuit board. The one or a plurality of circuit devices is, for example, an integrated circuit (IC), FPGA (field-programmable gate array), or the like. The one or a plurality of circuit elements is, for example, a resistor, a capacitor, or the like. The processor 30 may be implemented by a CPU (Central Processing Unit), GPU (Graphics Processing Unit), DSP (Digital Signal Processor), or the like.

The storage device 70 is a device for storing information, and functions as a storage section. The storage device 70 is a memory implemented, for example, by a semiconductor memory such as an SRAM (Static Random Access Memory), a DRAM (Dynamic Random Access Memory) or the like, or may also be a memory implemented by a magnetic storage device such as a register, a HDD (Hard Disk Device) or the like, or an optical storage device such as an optical disc device or the like. For example, the memory stores therein a computer-readable commands, and part or all of the functions of the sections of the information processing system 20 are achieved as processes with the processor 30 executing the commands. These commands may be a command set included in a program, or may be commands to give operating instructions to the hardware circuit of the processor 30. Furthermore, part or all of the sections of the information processing system 20 may be implemented by cloud computing.

The processor 30 includes a processing section 40. The processor 30 may also include a control section 50, a display device interface 60, and an endoscope interface 62. The processing section 40 performs a process of presuming route information of a lumen, a process of generating a display image, and the like. The control section 50 performs a control process of the electrically-driven endoscopic operation. These processes can be achieved by the hardware of the control device 600 in FIG. 21.

The display device interface 60 is a section for outputting the display image and performs an interface process with respect to the display device 90. For example, the display image data generated by the processor 30 is output to the display device 90 via the display device interface 60, and the display image is displayed on the display device 90. The endoscope interface 62 serves as an image acquisition section and performs an interface process with respect to the endoscope 100. Specifically, the endoscope interface 62 performs an interface process with an endoscope processor 108, which performs various processes with respect to the endoscope 100. For example, the processor 30 acquires an endoscope image captured by the endoscope 100 via the endoscope interface 62. In this case, the endoscope processor 108 performs various processes, such as image processing, with respect to the endoscope image. The endoscope processor 108 is implemented, for example, by the video control device 500 described later with reference to FIG. 21. The display device 90 may be implemented by, for example, a liquid crystal display device, an organic EL display, a CRT monitor or the like. EL stands for Electro Luminescence and CRT stands for Cathode Ray Tube. The details of the endoscope 100 are described later. In the following, the endoscope 100 may be described as a side-view type endoscope 100-A or a direct-view type endoscope 100-B, depending on the situation. Similarly, in the following, the distal end section 130 may be described as a side-view type endoscope distal end section 130-A or a direct-view type endoscope distal end section 130-B, depending on the situation.

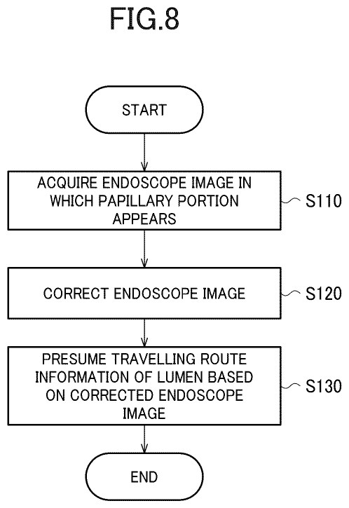

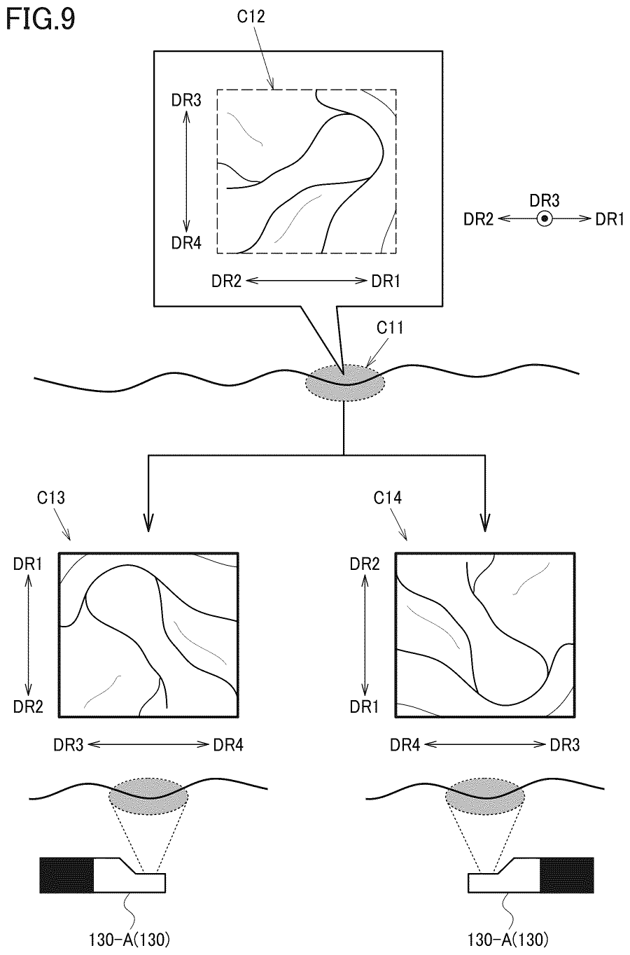

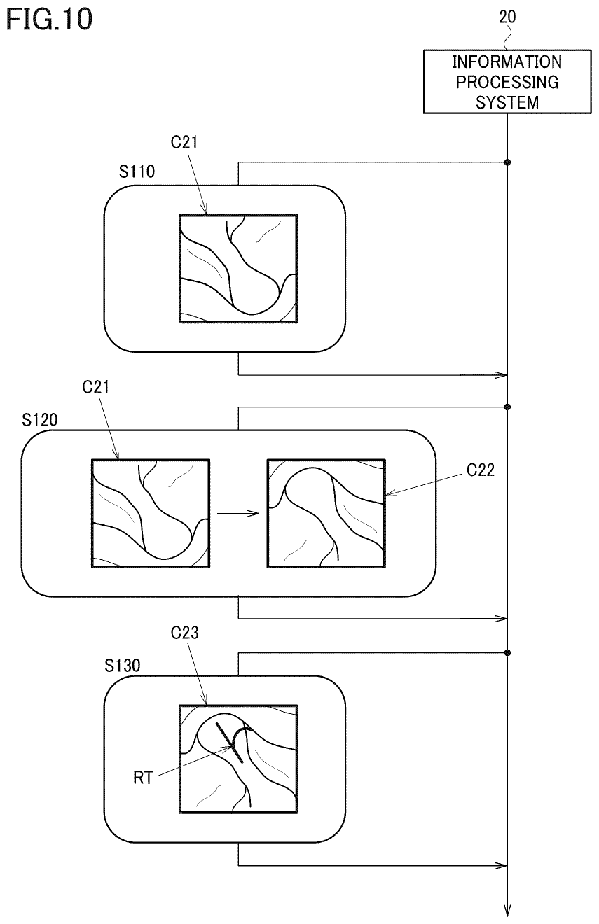

The method of the present embodiment is described below with reference to FIG. 8, FIG. 9, and FIG. 10. FIG. 8 is a flowchart explaining a process example of the present embodiment. FIG. 9 is an explanatory view of an example of a relationship between organ structure and endoscope image. FIG. 10 is an explanatory view of an example of endoscope image processed by the process example of FIG. 8.

The flowchart in FIG. 8 is explained below. The timing at which each step in FIG. 8 is performed may be determined automatically or manually by an operator. In FIG. 8, the processor 30 (the processing section 40, the same hereafter) including hardware acquires an endoscope image in which a papillary portion appears, from the endoscope 100 (step S110). For example, the processor 30 acquires an endoscope image captured by the imaging section included in the distal end section 130 via the endoscope interface 62. The processor 30 then performs a process of generating a display image to be displayed on the display device 90, based on the endoscope image acquired, for example, in the step S140 described later. The generated display image is output to the display device 90 by the display device interface 60 and is displayed on the display device 90.

For example, it is assumed that a papillary portion is present in the side wall of the lumen as shown in C11 in FIG. 9, and that the papillary portion has the structure shown in C12. In FIG. 9, the direction DR1 is assumed to be along the direction from the stomach side toward the duodenal side, and the direction DR2 is assumed to be along the direction opposite to the direction DR1, that is, the direction from the jejunal side toward the duodenal side. The directions DR3 and DR4 are assumed to be perpendicular to the direction DR1 and along the wall of the lumen, and the direction DR3 is assumed to go upward with respect to the paper surface. The distal end section 130 shown in FIG. 9 is assumed to be a side-view type endoscope distal end section 130-A. The endoscope image captured by the side-view type endoscope distal end section 130-A of the present embodiment is assumed to be captured with the distal end side of the side-view type endoscope distal end section 130-A facing upward.

As described above, if the organ structure has not been changed, the distal end of the side-view type endoscope distal end section 130-A is inserted toward the direction DR1. Then, as described above, it is assumed that the papillary portion shown in C11 and the imaging section (not shown) of the side-view type endoscope distal end section 130-A have a given positional relationship. In this case, the endoscope image captured by the imaging section is the image shown in C13 because the direction DR1 goes upward. On the other hand, if the organ structure has been modified, the distal end of the side-view type endoscope distal end section 130-A is inserted toward the direction DR2. If the imaging section of the side-view type endoscope distal end section 130-A inserted toward the direction DR2 can be positioned to directly face the papillary portion shown in C11, the endoscope image captured by the imaging section is the image shown in C14 because the direction DR2 goes upward. In other words, the endoscope image shown in C13 and the endoscope image shown in C14 are in a relationship of 180-degree rotation around an axis perpendicular to the endoscope image. In the following explanation, an action such as rotating an image around an axis perpendicular to the image is simply referred to as rotating the image, or the like.

If the process of FIG. 8 is performed on a modified organ structure, the processor 30 acquires the endoscope image shown in C21 in FIG. 10. The endoscope image shown in C21 in FIG. 9 corresponds to the endoscope image shown in C14 in FIG. 8.

The explanation continues below with reference back to FIG. 8. Thereafter, the processor 30 corrects the endoscope image (step S120). Specifically, the processor 30 corrects the endoscope image based on correction information, which is identified by the organ structure. The correction information is, for example, such that the processor 30 reads out information, such as a medical record, stored in the storage device 70, and carries out a search as to whether or not a treatment based on the Billroth II method described above or the like has been performed. Then, if the processor 30 detects that the Billroth II method or the like has been performed, the processor 30 identifies modification of organ structure. In this case, the processor 30 performs a process of correcting the endoscope image acquired in the step S110.

Specifically, the endoscope image shown in C21 in FIG. 10 is corrected to the endoscope image shown in C22 by the step S120 in FIG. 8. The endoscope image shown in C22 is the same as the endoscope image shown in C13 in FIG. 9. Specifically, the step S120 in FIG. 8 corrects the endoscope image shown in C21 to the endoscope image shown in C22 by performing a process of rotating the image 180 degrees. In other words, by the step S120 in FIG. 8, the endoscope image that was captured in a way different from normal due to the modification of the organ structure is corrected to an image equivalent to the endoscope image that would have been captured when the papillary portion and the imaging section of the side-view type endoscope distal end section 130-A had a given positional relationship.

Thereafter, as the step S130, a process of presuming (determining) the route information of a lumen, which is at least one of the biliary duct and the pancreatic duct, is performed based on the corrected endoscope image. The route information of a lumen is information to identify the route of the biliary duct or the pancreatic duct. The route information may be, for example, information to identify one of the route classification patterns described later, or direction information, position information, shape information or the like of the lumen to identify the route of the lumen. The step S130 may be a process in which the processor 30 determines one of the route classification patterns as described later, or may be a combination of a process in which the processor 30 enumerates a plurality of route classification patterns and a process in which the operator selects one classification pattern based on his/her experience. For example, the processor 30 may also inform the operator of the presumed route information by voice. Further, for example, the processor 30 may also perform a process of displaying the image shown in C23. The image shown in C23 is an image in which the lumen route image RT serving as the route guide image, which is described later, is superimposed on the endoscope image in C22.

Based on the above, the information processing system 20 of the present embodiment includes the processor 30 that includes hardware. The processor 30 acquires an endoscope image in which a papillary portion appears, from the endoscope 100, corrects the endoscope image based on the correction information which is identified by the organ structure, and presumes the route information of a lumen based on the corrected endoscope image.

In this way, since the information processing system 20 of the present embodiment acquires from the endoscope 100 an endoscope image in which a papillary portion appears, the operator can perform a treatment such as ERCP or the like using the endoscope 100. In addition, since the endoscope image is corrected based on the correction information identified by the organ structure, when the positional relationship of the distal end section of the endoscope with respect to the papillary portion is different because of modification of organ structure, it is possible to acquire an endoscope image equivalent to that when performing ERCP or the like before the modification of organ structure. Further, since the route information of a lumen is presumed based on the corrected endoscope image, the route of the lumen can be accurately presumed based on past cases, experiences, or the like. This allows the information processing system 20 to appropriately assist the operator in performing a treatment such as ERCP or the like when the organ structure is modified. In this regard, the aforementioned U.S. Patent Application Publication No. 2017/0086929 does not disclose a method for assisting a treatment such as ERCP or the like when the organ structure is modified.

Further, the method of the present embodiment may also be realized as a medical system 10, which is described later with reference to FIG. 21. Specifically, the medical system 10 of the present embodiment includes the information processing system 20 described above, and the endoscope 100. In this way, the same effects as those described above can be achieved.

Further, the method of the present embodiment may also be realized as an information processing method. Specifically, the information processing method of the present embodiment includes a step (step S110) of acquiring an endoscope image in which a papillary portion appears, from the endoscope 100, and a step (steps S120 and S130) of correcting the endoscope image based on the correction information identified by the organ structure and presuming the route information of a lumen based on the corrected endoscope image. In this way, the same effects as those described above can be achieved.

Further, in the information processing system 20 of the present embodiment, the processor 30 may correct the endoscope image to an image in which the imaging section of the endoscope 100 and the papillary portion have a given positional relationship. In this way, it is possible to acquire an endoscope image that is equivalent to the endoscope image captured when the treatment is performed before the organ structure is modified.

Further, in the information processing system 20 of the present embodiment, the processor 30 may correct the endoscope image by a correction process that includes image rotation correction and presume the route information of lumen based on the corrected endoscope image. In this way, it is possible to acquire an image equivalent to the endoscope image captured when the treatment is performed before modification of organ structure by the correction process including image rotation correction.

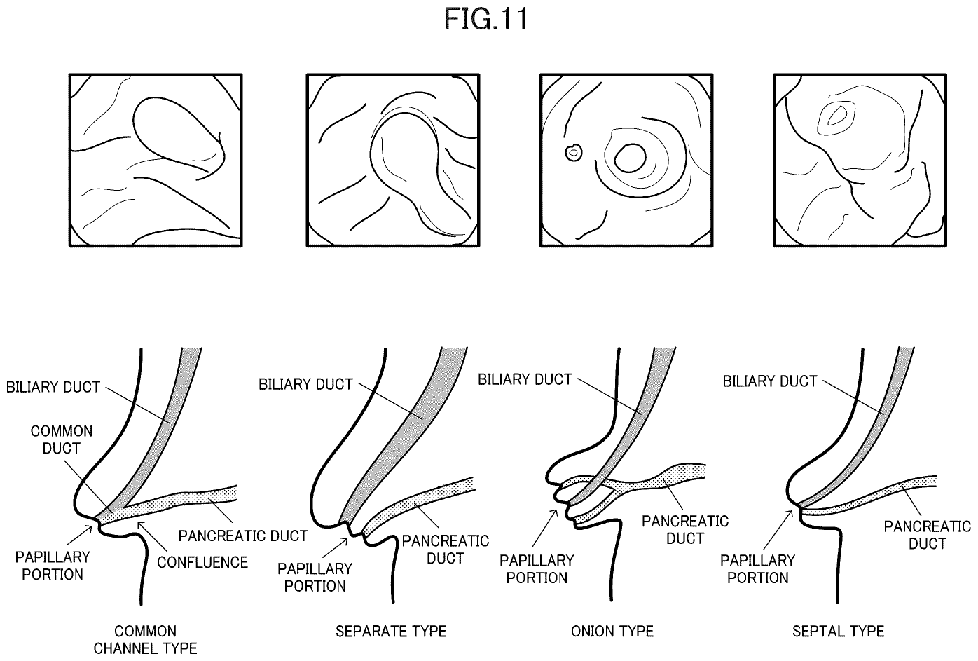

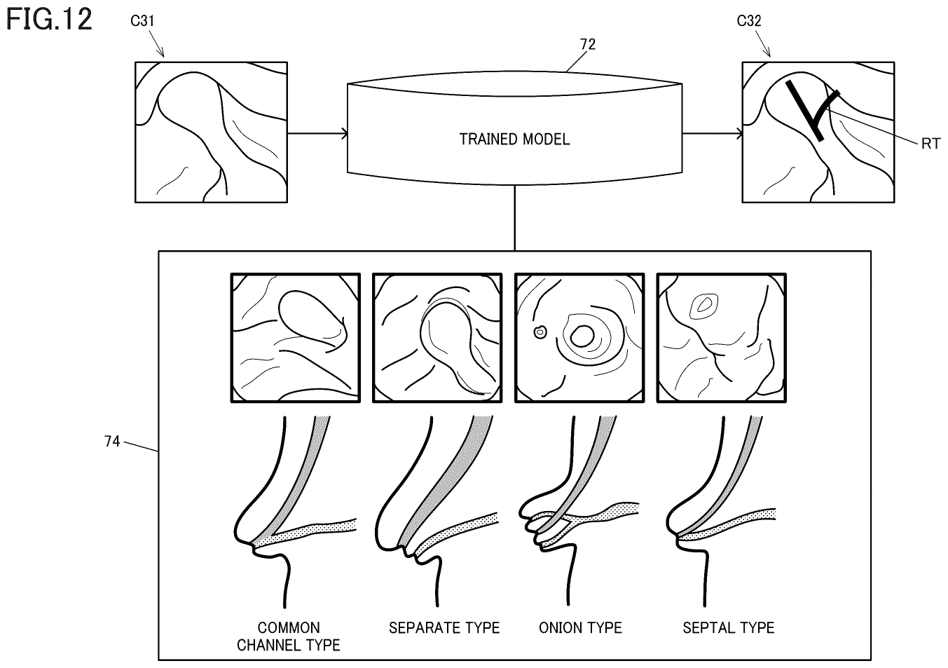

The lumen route image RT is image data prepared in advance based on the following classification patterns, which are described later with reference to FIG. 12 in details. The classification patterns are those listed in the classification model as an attempt to classify the relationship between the form of the papillary portion and a route of biliary duct or the like into a plurality of patterns based on past cases and experiences of the operator. For example, FIG. 11 shows examples of classification patterns of the papillary portion and the endoscope images observed in the classification patterns. The classification patterns of the routes of the biliary duct and the pancreatic duct include, for example, the common channel type, the separate type, the onion type, and the septal type, as shown in FIG. 11. In the common channel type, the biliary duct and the pancreatic duct merge into a common duct at the confluence thereof, and the common duct opens to the papillary portion. In the separate type, the biliary duct and the pancreatic duct are separately open to the papillary portion and there is no confluence or common duct. In the onion type, the pancreatic duct is branched into two parts, and the biliary duct opens in the center of the opening of the two branched pancreatic ducts. In the septal type, the biliary duct and the pancreatic duct open to the papillary portion at their confluence, and there is no common duct. The common channel type is most common among the classification patterns of the papillary portion in patients; however, there are also patients having the separate type, the onion type, and the septal type. The classification patterns in the present embodiment are not limited to those in FIG. 11, and classification patterns based on various types of classification models as classification patterns of the opening form of the papillary portion, for example, those classified into individual type, nodular type, villous type, flat type, and longitudinal type, can be used.

The presumption of the classification pattern of the papillary portion involved in the step S130 is performed, for example, by a trained model 72. For example, as shown in FIG. 7, the storage device 70 stores the trained model 72 trained to output the route information of a lumen, which is at least one of the biliary duct and the pancreatic duct, with respect to the endoscope image. The processor 30 then presumes the route information from the endoscope image by the process based on the trained model 72. More specifically, the processor 30 inputs the endoscope image shown in C22 in FIG. 10 to the trained model 72, and displays the image shown in C23 in FIG. 10 as a result of presumption of the route information of a lumen based on the output information from the trained model 72.

The trained model 72 has been trained by machine learning using training data 74, and is implemented by, for example, a neural network (not shown) or the like. The neural network includes an input layer to which input data is entered, an intermediate layer for performing a calculation process with respect to the data entered via the input layer, and an output layer for outputting a recognition result based on the calculation result output from the intermediate layer. For example, the trained model 72 has been trained using the training data 74, which is a data set in which input data and correct answer data are associated with each other. For example, the storage device 70 stores a program that describes an inference algorithm and parameters used for the inference algorithm, as the information of the trained model 72. The processor 30 then executes the process of presuming the route information of a lumen based on the endoscope image by executing the program using the parameters stored in the storage device 70. As the inference algorithm, for example, the aforementioned neural network can be used, and the weight coefficients of the inter-node connections in the neural network serve as the parameters of the inference algorithm. The inference algorithm is not limited to a neural network, and various types of machine learning process for use in recognition process may be used.

FIG. 12 is a diagram conceptually explaining the process using the trained model 72. The trained model 72 is generated by a training device (not shown) during the training phase. The training device stores untrained models with initial weight coefficients in a storage device (not shown). The training data 74 is input to the untrained model, and feedback is given to the untrained model based on the inference result, thereby optimizing the weight coefficient. The trained model 72 is thus generated. The training data 74 contains a plurality of data sets, each of which contains input data and correct answer data. The input data is the endoscope image captured when the imaging section of the endoscope 100 and the papillary portion have a given positional relationship as mentioned above. The correct answer data is classification patterns including the common channel type, the separate type, the onion type, and the septal type. A large number of these data sets can be prepared. This is because there are many cases captured when the imaging section of the endoscope 100 and the papillary portion have a given positional relationship.

Further, in the inference phase, when the endoscope image shown in C31, which is the input data, is input to the trained model 72 using, for example, the endoscope interface 62 as an input section, the image shown in C32, which corresponds to the correct answer data, is output as the output data from the trained model 72 using the display device interface 60 as an output section. This inference phase corresponds to the step S130. The endoscope image shown in C31 in FIG. 12 corresponds to the endoscope image shown in C22 in FIG. 10, and the image shown in C32 in FIG. 12 corresponds to the image shown in C23 in FIG. 10.

The lumen route image RT is an image by which the operator can visually recognize what kind of lumen route the biliary duct and the pancreatic duct have. Thus the lumen route image RT serves as a marker image showing the routes of the biliary duct and the pancreatic duct. More specifically, for example, if the endoscope image shown in C31 is an endoscope image having common channel type features, the lumen route image RT is displayed as an image including the biliary duct, the pancreatic duct, and the common duct. Similarly, although the illustrations are omitted hereafter, if the classification pattern is the separate type, the lumen route image RT is displayed as an image in which the biliary duct and the pancreatic duct are separated. When the classification pattern is the onion type, the lumen route image RT is displayed as an image with one biliary duct and two pancreatic ducts. If the classification pattern is the septal type, the lumen route image RT is displayed as an image in which the biliary duct and the pancreatic duct merge and there is no common duct.

The trained model 72 may also be trained to directly output the lumen route image RT as output data. In this case, the region corresponding to the lumen route image RT is segmented with respect to the endoscope image by semantic segmentation or the like using the trained model 72 by way of CNN (Convolutional Neural Network) or the like.

In view of the above, the information processing system 20 of the present embodiment includes the storage device 70 serving as a memory for storing the trained model 72 that is trained by the training data 74, which is a data set including a training endoscope image as input data and route information of a lumen as correct answer data. The processor 30 inputs the corrected endoscope image to the trained model 72, thereby presuming the route information of the lumen. In this way, it is possible to more accurately presume the route information of lumen using the trained model 72. This allows for display of a route guide image that enables more appropriate guide of lumen route. This provides an assistance to the operator during the ERCP procedures. For example, by viewing the lumen route image RT, the operator can visually identify what kind of lumen route the biliary duct and the pancreatic duct have. Specifically, for example, the operator can confirm the route of the biliary duct or the pancreatic duct in the back of the opening in the endoscope image showing the papillary portion, such as that shown in FIG. 3, with reference to the lumen route image RT, thereby enabling intubation of the treatment tool 400, such as a cannula, from the opening. This allows even inexperienced operators and the like to easily perform the procedure of ERCP using the lumen route image RT as a guide.

Further, in the information processing system 20 of the present embodiment, the trained model 72 may be trained by the training data 74 based on the training endoscope image captured when the imaging section of the endoscope 100 and the papillary portion have a given positional relationship. In this way, it is possible to generate the trained model 72 that has been trained by machine learning based on the endoscope image captured under conditions equivalent to those of a normal treatment.

Further, in the information processing system 20 of the present embodiment, the trained model 72 may be trained by the training data 74 based on the classification pattern of the papillary portion. In this way, an appropriate lumen route presumption process can be performed in accordance with the classification pattern of the papillary portion. This allows for appropriate generation of route guide images that reflect the classification patterns.

Because there are less cases with modified organ structures, which are described above in FIG. 4 to FIG. 6, it is difficult to construct the trained model 72 that has been trained by machine learning using, as the data set, endoscope images captured when the organ structure has been modified. In this regard, by applying the method of the present embodiment, endoscope images captured when the organ structure has been modified can be corrected to those equivalent to endoscope images captured when the imaging section of the endoscope 100 and the papillary portion have a given positional relationship, and can be used as input data for the trained model 72 that can be constructed based on past cases.

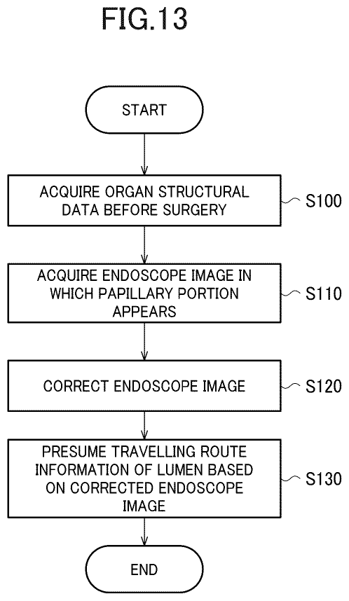

The method of the present embodiment may also be performed, for example, according to the process example shown in the flowchart in FIG. 13. The flowchart in FIG. 13 differs from the flowchart in FIG. 8 in that the processor 30 further performs a process (step S100) of acquiring organ structural data before surgery. Further, in the step S120, the processor 30 determines correction information based on the structural data before surgery acquired in the step S100, and performs a process of correcting the endoscope image based on the correction information. The organ structural data before surgery is image information in which the position of each pixel, which is acquired in advance by, for example, CT (Computerized Tomography), MRI (Magnetic Resonance Imaging), PET (Positron Emission Tomography) or the like before the treatment of the present embodiment is performed, is defined by a three-dimensional coordinate system.

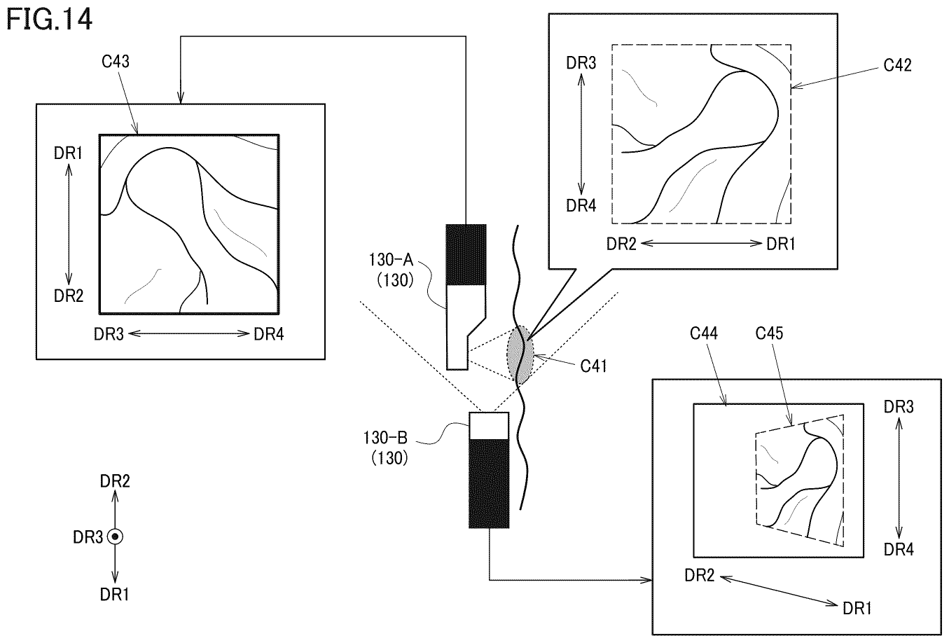

The correction information based on the structural data before surgery can be used, for example, in the case shown in FIG. 14. Although FIG. 14 illustrates the side-view type endoscope distal end section 130-A and the direct-view type endoscope distal end section 130-B together for ease of explanation, the illustration does not specify that the side-view type endoscope distal end section 130-A and the direct-view type endoscope distal end section 130-B are inserted simultaneously in a single treatment.

The example shown in FIG. 14 differs from the example in FIG. 8 in that the distal end of the direct-view type endoscope distal end section 130-B is inserted toward the direction DR2 when the organ structure is modified. For example, it is assumed that a papillary portion is present on the side wall of the lumen shown in C41 in FIG. 14, and that the papillary portion has the structure shown in C42. When the papillary portion shown in C41 and the imaging section of the side-view type endoscope distal end section 130-A are in a given positional relationship, the endoscope image captured by the imaging section will be the image shown in C43, as in the case of FIG. 8.

When the direct-view type endoscope 100-B is used in FIG. 14, it is generally difficult to position the imaging section of the direct-view type endoscope distal end section 130-B to directly face the papillary portion shown in C41. In other words, in FIG. 14, it is generally difficult to make the orientation of the optical axis of the imaging section of the direct-view type endoscope distal end section 130-B to be in parallel with the orientation of the optical axis of the imaging section of the side-view type endoscope distal end section 130-A when the imaging section of the side-view type endoscope distal end section 130-A and the papillary portion have a given positional relationship. Therefore, the imaging section of the direct-view type endoscope distal end section 130-B captures an image of the papillary portion shown in C41 from an oblique direction. In other words, the organ structure is modified, and the endoscope image captured by the direct-view type endoscope 100-B will be, for example, the image shown in C44. In the endoscope image shown in C44, the papillary portion shown in C41 is distorted as shown in C45 because the region near the imaging section is displayed larger and the region far from the imaging section is displayed smaller.

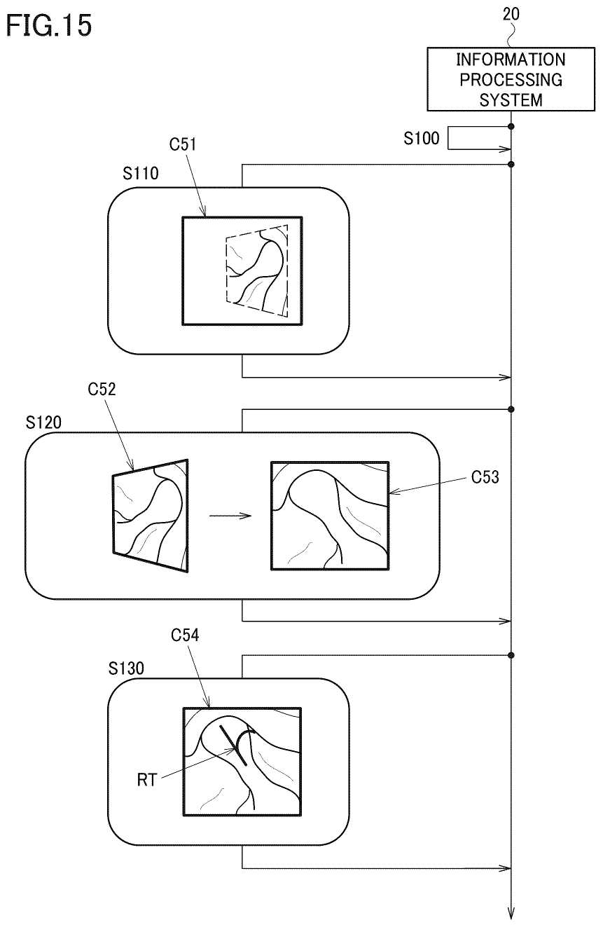

FIG. 15 shows a flow of changes in images when the process of FIG. 13 is applied in the situation shown in FIG. 14. After the step S100 is performed, the processor 30 acquires the endoscope image shown in C51 in FIG. 15 in the step S110. The endoscope image shown in C61 corresponds to the endoscope image shown in C45 in FIG. 14.

The processor 30 then corrects the endoscope image shown in C51 so that an image equivalent to the endoscope image captured when the imaging section of the side-view type endoscope distal end section 130-A and the papillary portion have a given positional relationship can be acquired by the step S120, as in the case described above in FIG. 10. Specifically, the processor 30 performs a first process of extracting the image shown in C52 from the endoscope image acquired in C51, and a second process, which is inclination correction with respect to the image shown in C52 obtained by the first process. In the case of FIG. 14, the processor 30 further performs a third process to perform rotation correction with respect to the image having been corrected by the inclination correction. The first process is, for example, a process in which the operator trims the image of the portion shown in C52 from the endoscope image shown in C51, and this process can be achieved using known methods.

The inclination correction herein refers to correcting the image as if the line-of-sight direction of the imaging section was changed. Specifically, for example, by the inclination correction, an image captured with the papillary portion in a non-front view (as described above) is corrected as if the image was captured with the papillary portion in a front view. Specifically, the inclination correction involved in the second process can also be referred to as tilt-distortion correction. Since many methods have been proposed for tilt-distortion correction, and all of them are fully publicly known, detailed descriptions thereof are omitted here. The following method can be referred to as an example. For example, the processor 30 first determines a first vector, which is the direction vector along the optical axis of the imaging section of the side-view type endoscope distal end section 130-A when the imaging section of the side-view type endoscope distal end section 130-A and the papillary portion have a given positional relationship. The processor 30 then determines a second vector, which is the direction vector along the optical axis of the imaging section of the direct-view type endoscope distal end section 130-B when the imaging section of the direct-view type endoscope distal end section 130-B observes the papillary portion in the best direction. The best direction refers to a direction of the optical axis of the imaging section when the papillary portion is imaged as close as the front view in the field of view of the imaging section (not shown) of the direct-view type endoscope distal end section 130-B. The first and second vectors can be obtained, for example, by a method of constructing three-dimensional shape information of structural data after the organ structure has been modified using volume rendering or other methods, and simulating the position information of the papillary portion, a method of simulating how the direct-view type endoscope distal end section 130-B comes closer to the papillary portion, and the like.

The processor 30 then determines the angle between the plane perpendicular to the first vector and the plane perpendicular to the second vector, as well as the distance between the imaging section of the direct-view type endoscope distal end section 130-B and the papillary portion, and performs a process of appropriately enlarging the image displayed smaller on the back side based on the determined angle and distance. In this way, the tilt-distortion of the image shown in C52 is corrected. Thus, the structural data acquired by the processor 30 in the step S100 is the data necessary to determine the parameters for correcting the distortion of the image shown in C45. The step S120 may, for example, perform the second process on the entire endoscope image shown in C51 and, as the first process, trim an image of a portion corresponding to the image shown in C53 from the resulting image.

Further, in the case of FIG. 14, the image shown in C52 corrected by the second process corresponds to the image shown in C45 in FIG. 14, and therefore this image is an image with the direction DR3 directing upward. However, the endoscope image captured when the side-view type endoscope distal end section 130-A and the papillary portion have a given positional relationship is an image with the direction DR1 directing upward as shown in C43 in FIG. 14. Therefore, as the third process, the image obtained by the second process is further corrected by rotating it 90 degrees to the left. Based on the above, by the step S120, the image shown in C52 captured by the imaging section of the direct-view type endoscope distal end section 130-B is corrected to the image shown in C53, which is an image equivalent to the endoscope image captured when the side-view type endoscope distal end section 130-A and the papillary portion have a given positional relationship. This allows the operator to view an endoscope image corresponding to the image of the papillary portion that the operator knows from experience, thus easily predicting the lumen route. Alternatively, it can be used as input data for the trained model 72. If the image acquired in the step S110 is an image with the direction DR1 directing upward, as with the endoscope image captured when the side-view type endoscope distal end section 130-A and the papillary portion have a given positional relationship, the third process is omitted in the step S120. The processor 30 may display both the endoscope image acquired in the step S110 and the image corrected in the step S120 on the display device 90.

The subsequent step S130 is performed in the same manner as in FIG. 10, and the lumen route image RT as a route guide image is superimposed on the image shown in C53 as shown in C54.

Based on the above, in the information processing system 20 of the present embodiment, the processor 30 acquires organ structural data before surgery, and determines correction information based on the structural data. In this way, the parameters necessary to correct the endoscope image captured when the organ structure is modified can be acquired.

Further, in the information processing system 20 of the present embodiment, the processor 30 may correct the endoscope image by a correction process that includes image inclination correction and may presume the route information of lumen based on the corrected endoscope image. In this way, when the imaging section of the distal end section 130 cannot capture an image from a given angle with respect to the papillary portion, by performing the correction process including inclination correction, an image equivalent to the image captured when the imaging section of the distal end section 130 and the papillary portion have a given positional relationship can be acquired.

As is clear in comparison of FIG. 1 with FIGS. 6 to 8, the route to the papillary portion becomes more complicated after a surgical treatment, such as the aforementioned Billroth II method, RYGB, or the like, compared with before these surgical treatments. Therefore, when ERCP is performed on a patient who has gone through such a surgical treatment, in some cases, insertion of the direct-view type endoscope distal end section 130-B into the papillary portion may become finally possible by using the direct-view type endoscope 100-B. In such a situation, it becomes difficult to capture an image equivalent to those captured when the side-view type endoscope distal end section 130-A and the papillary portion have a given positional relationship, as in the normal ERCP cases. More specifically, since it is difficult to bring the imaging section of the direct-view type endoscope distal end section 130-B to directly face the papillary portion, the imaging section can only capture an image from an oblique direction with respect to the papillary portion; as a result, the papillary portion appears distorted in the acquired endoscope image. Therefore, when performing the ERCP treatment on a patient who has gone through such a surgical treatment, it is difficult to presume the route of a lumen from the acquired endoscope image.

In this regard, by applying the method of the present embodiment, when the ERCP treatment is performed on a patient who has gone through such a surgical treatment, the acquired endoscope image can be corrected so that the lumen route can be presumed. This provides an assistance to the operator who performs the ERCP treatment on a patient who has gone through such a surgical treatment so that the operator can more easily perform the treatment.

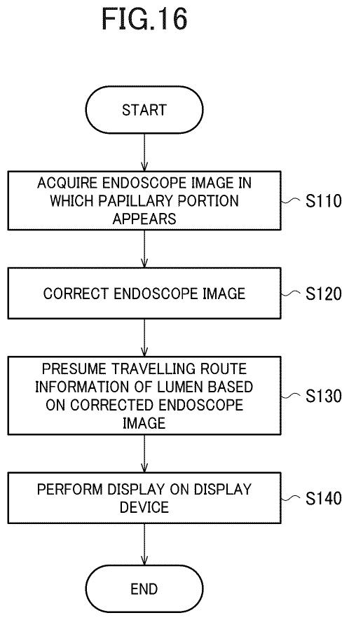

The method of the present embodiment may also be performed, for example, according to the process example shown in the flowchart in FIG. 16. FIG. 16 is a flowchart in which the step S140 is further added to the flowchart of FIG. 8. FIG. 16 may also be a flowchart in which the step S140 is further added to the flowchart of FIG. 13. The step S140 is a process of displaying a display image on the display device 90. The step S140 can be performed in various different ways, including, for example, the steps S140-A and S140-B, which are described later in detail, or other processes may also be added.

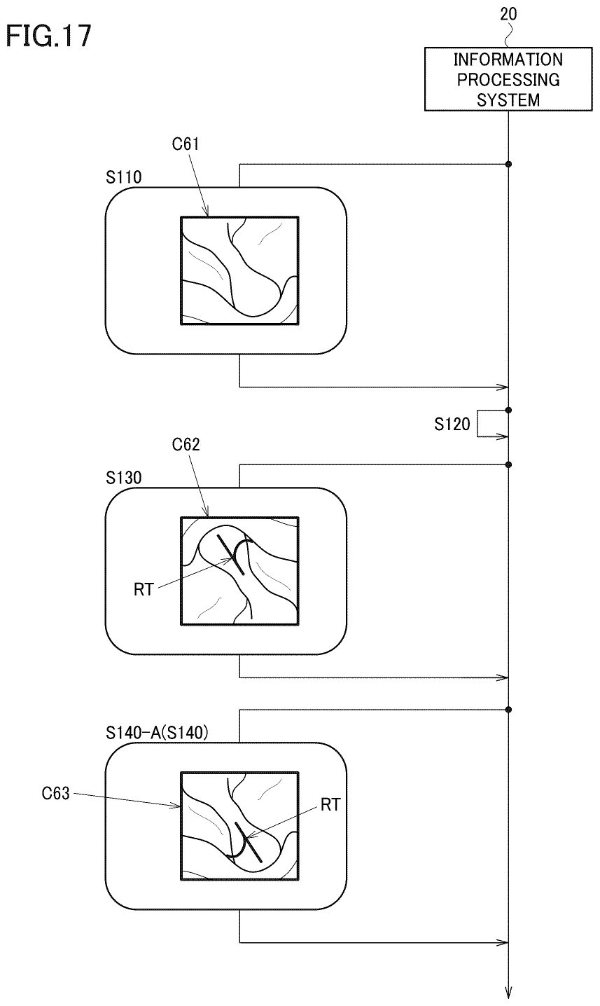

FIG. 17 shows a flow of changes in endoscope images when the process of FIG. 16 is applied in the example shown in FIG. 9. For example, the processor 30 acquires the image shown in C61 by the step S110. The route of the lumen is then presumed in the step S130, and an image on which RT is superimposed is generated as shown in C62.

Thereafter, in the step S140, the processor 30 performs a correction process of rotating the image shown in C62 by 180 degrees, and then performs a process of displaying the image shown in C63 on the display device 90 as the display image. In other words, the processor 30 performs a process of re-correcting the uncorrected endoscope image shown in C61. Also, the lumen route image RT generated in the step S130 is corrected to correspond to the corrected image by the step S140. Specifically, although the processor 30 corrects the endoscope image as shown in C62 by the step S130, the operator perceives, through the display device 90, the image shown in C63 in which the lumen route image RT is superimposed on the uncorrected endoscope image shown in C61. As described above, in the information processing system 20 of the present embodiment, the processor 30 generates a display image in which a route guide image to provide guidance to a route of a lumen leading to the papillary portion is superimposed on an uncorrected endoscope image based on the presumption result of the route information of the lumen, and display the display image on the display device 90. For example, if the endoscope image is automatically corrected during a treatment, the operator may be confused. In this regard, by applying the method of the present embodiment, it is possible to make the display appear that only the route guide image is superimposed on the endoscope image, thus preventing any confusion for the operator. Further, in the information processing system 20 of the present embodiment, the processor 30 may also correct the route guide image so that the route guide image corresponds to the correction of the endoscope image. In this way, when the endoscope image is corrected after the route guide image is displayed, the route guide image can be placed in an appropriate position in the corrected endoscope image.

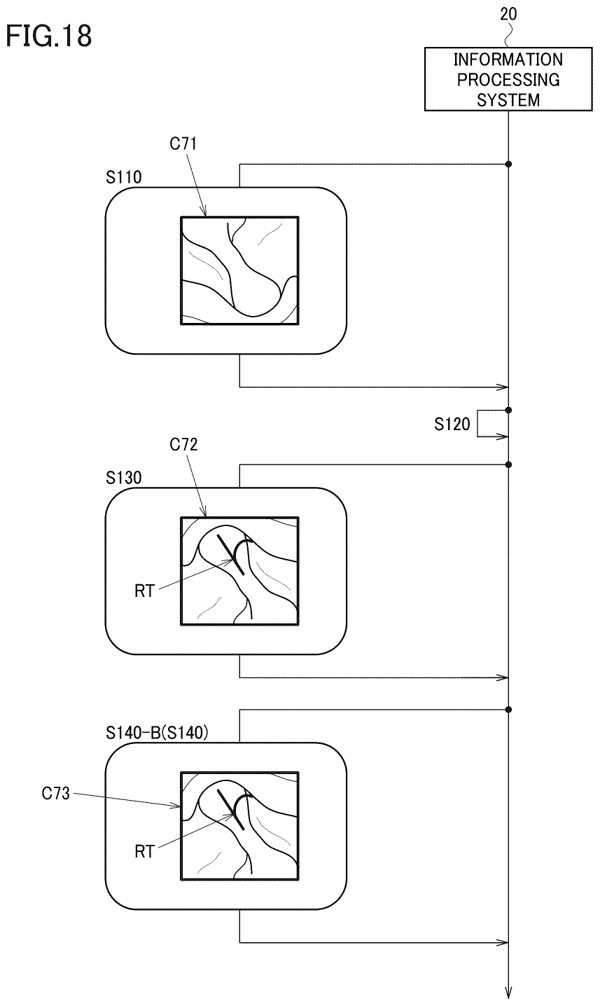

The step S140 may also be performed as a process of displaying the lumen route image RT and the endoscope image corrected in the step S130 on the display device 90 as they are as display images. In this case, this process is referred to as a step S140-B. For example, as shown in FIG. 18, the process is the same as FIG. 17 in that the processor 30 acquires the image shown in C71 by the step S110, presumes the route of the lumen in the step S130, and generates an image on which the lumen route image RT is superimposed as shown in C72. Thereafter, the processor 30 also causes the display device 90 to display, as the display device, the image shown in C73 in the same manner as for the image shown in C72 by the step S140. As described above, in the information processing system 20 of the present embodiment, the processor 30 generates a display image in which a route guide image that provides guidance to a route of a lumen leading to the papillary portion is superimposed on the corrected endoscope image based on the presumption result of the route information of the lumen, and displays the display image on the display device 90. In this way, the endoscope image and the route guide image with the imaging section of the endoscope and the papillary portion in a given positional relationship can be presented to the operator. For operators who have seen many endoscope images captured with the imaging section of the endoscope and the papillary portion in a given positional relationship, it may be easier to perform a treatment with an image that is closer to the image they are familiar with. In such cases, it is more convenient to display the image corrected in the step S130 as is on the display device 90.

Further, for example, when the endoscope 100 is controlled electrically as described later, before the step S140-B is performed, a display to announce to the operator or the like whether or not to correct the endoscope image when it is displayed may be performed. It is also possible to arrange such that the step S140-A and the step S140-B are switchable as appropriate. It is also possible to arrange such that both the image displayed by the step S140-A and the image displayed by the step S140-B can be displayed.

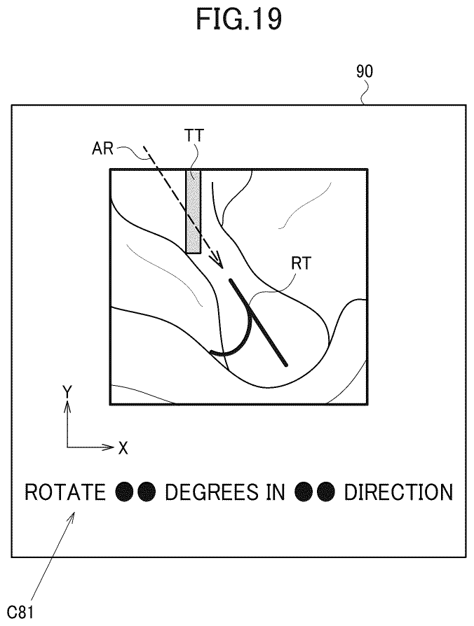

Further, in the step S140, the content of the operation effective for the cannulation step may be presumed, and a process of navigating the content of the operation to the operator may also be performed. For example, as described above in FIG. 17, as the processor 30 performs the step S140-A, the lumen route image RT is superimposed on the endoscope image in the display image. Further, as shown in FIG. 19, the direction image AR may further be superimposed thereon. The direction image AR is an image showing the target direction of the treatment tool 400, determined in consideration of, for example, the classification pattern of the papillary portion presumed in the step S130. Specifically, in the example in FIG. 19, the lumen route image RT and the direction image AR correspond to the route guide image.

For example, in the cannulation step, the treatment tool image TT is further superimposed on the endoscope image on the display device 90 because the treatment tool 400 is headed toward the papillary portion. At this time, the processor 30 may navigate to correct the direction in which the treatment tool 400 is headed as shown in C81 if the direction in which the treatment tool 400 is headed does not match the direction indicated by the direction image AR. For example, after detecting the treatment tool image TT, the processor 30 determines the inclination of the approximation straight line obtained by approximating the treatment tool image TT to a straight line, and displays the angle between the inclination of the approximation straight line and the inclination of the direction image AR on the display device 90. In response to this, the operator considers, for example, changing the angle of the desk stand (not shown) included in the distal end section 130 or changing the angle of the bending section of the treatment tool 400, so as to modify the angle. The operator may also consider, for example, changing the angle of the bending section 102, which is described below, or retracting the insertion section 110 so that the distal end section 130 once becomes distant from the vicinity of the papillary portion. In this case, the image of the papillary portion must be captured again; therefore, the positioning step shown in FIG. 2 and related processes are performed again.

The example shown in C81 is an example of navigation to correct the rotation angle. The navigation may also be performed using, for example, the coordinates with the X and Y axes, which are the coordinate axes on the screen of the display device 90. In addition, regarding the description above that the step S140-A and the step S140-B may be made switchable as appropriate, in this case, the information for the navigation in C81 may also be made switchable. For example, if the endoscope image is corrected by being rotated 180 degrees by the step S140-B when the values of the X and Y coordinates are displayed as (+α, +β) in the navigation display shown in C81, a process of switching the coordinate display in the navigation display to (−α, −β) is performed.

Further, although not shown in FIG. 19, in predetermined cases, the processor 30 may, for example, display, on the display device 90, a navigation display to instruct temporary separation of the distal end section 130 from the papillary portion. Such a predetermined case refers to, for example, a case where it is clear that the angle between the inclination of the approximation straight line obtained by the processor 30 through approximation and the inclination of the direction image AR is larger than a certain angle, thus failing to appropriately correcting the direction in which the treatment tool 400 is headed by changing the angle of the desk stand or the angle of the bending section.

Further, the route of the lumen may also be presumed using, for example, ultrasound images obtained by an ultrasound endoscope (not shown) and endoscope images. Since the ultrasound endoscope is publicly known, description of its configuration and the like are omitted. Although the ultrasound image is not shown in the figure, the ultrasound image is assumed to be so-called a B-mode image. For example, although not shown in the figure, instead of the classification patterns shown in FIG. 11, etc., an ultrasound image showing the route of the lumen near the papillary portion may be associated with an endoscope image of the papillary portion captured when the ultrasound image is obtained. Further, the associated ultrasound and endoscope images may be used as training data 74 for machine learning. In other words, the trained model 72 shown in FIG. 12 may be constructed by performing machine learning using a data set including endoscope images as input data and ultrasound images as correct answer data. Since EUS-FNA and the like are often performed prior to ERCP, many ultrasound and endoscope images can be acquired, and these image data can be used as training data for the trained model 72. EUS-FNA stands for Endoscopic UltraSound-guided Fine Needle Aspiration.

The lumen route image RT may be created in advance so that it corresponds to the lumen route displayed in the ultrasound image, which is the correct answer data, and the lumen route image RT as the route guide image may be displayed by being superimposed on the endoscope image as a presumption result in the step S130 described above. Specifically, for example, in the inference phase, the endoscope image captured during the ERCP treatment may be used as input data, and the ultrasound image captured during the EUS-FNA or the like previously performed may be used as metadata, and these data may be input to the trained model 72, and an image in which the lumen route image RT is superimposed on the endoscope image may be output as output data. Based on the above, in the information processing system 20 of the present embodiment, the trained model 72 is trained by the training data 74 based on ultrasound images. In this way, a system for presuming the route of a lumen using ultrasound images can be constructed.

When the EUS-FNA described above or the like is performed, the medical system 10 described later may be used. Specifically, each componential unit of the ultrasound endoscope may be driven and controlled by electrical driving.

Further, the route information of a lumen may be presumed using an MRCP image obtained by MRCP and the endoscope image described above. MRCP stands for Magnetic Resonance Cholangio Pancreatography, which is an examination in which the gallbladder, the biliary duct, and the pancreatic duct are simultaneously extracted by an MRI examination device (not shown). MRCP images are acquired in advance by being captured by the MRI examination device before the ERCP treatment is performed.

For example, although the flowchart and other illustrations are omitted, in the step S110 mentioned above, the processor 30 performs a process of acquiring MRCP images stored in a storage device (not shown) of the MRI examination device and a process of acquiring the endoscope image mentioned above. Alternatively, it is also possible to perform a process of storing the MRCP images in the storage device 70 in advance, and retrieving the MRCP images from the storage device 70 in the step S110.

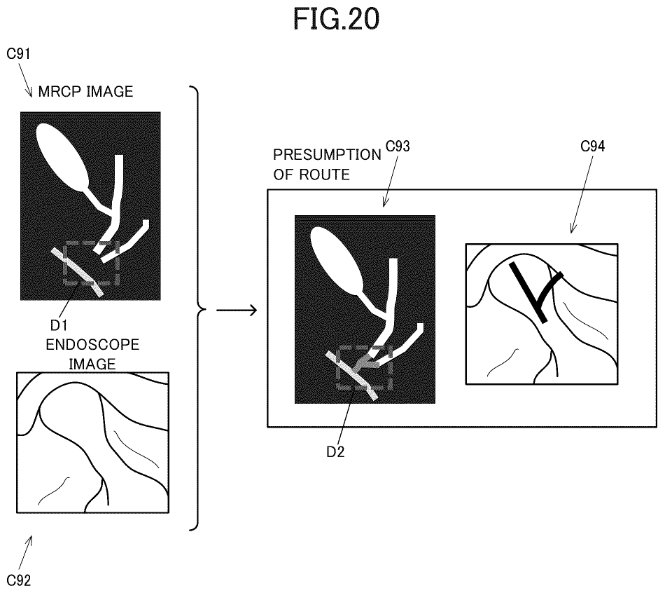

For example, as shown in D1, the MRCP image shown in C91 in FIG. 20 has a characteristic in that an image of a lumen near the papillary portion cannot be clearly captured. The region of D1 will be hereinafter referred to as an unclear region. The processor 30 then presumes the route of the lumen based on the endoscope image by the step S130 described above, and also performs a process of superimposing an image corresponding to the presumed route on the unclear region of the MRCP image as shown in D2. In this way, the route of the lumen in the unclear region of the MRCP image can be complemented. For example, when the presumed classification pattern is the common channel type, if the entire MRCP image looks natural as a result of superimposing the image corresponding to the common channel type on the unclear region of the MRCP image as shown in C93, the presumption accuracy of the trained model 72 is considered high.

The MRCP image may also be used as the training data 74 for the trained model 72. Since a large number of MRCP images can be acquired, they can be used as the training data 74 for machine learning. Specifically, the trained model 72 is constructed by performing machine learning with respect to an untrained model using a data set including the classification pattern of the papillary portion as the correct answer data and the MRCP image shown in C91 and the endoscope image shown in C92 as input data, and optimizing the weight coefficients. In addition to the endoscope interface 62 mentioned above, the interface for acquiring the MRCP images also functions as the input section during the inference phase in this case. Further, as shown in C94, the endoscope image on which, for example, an image such as the lumen route image RT corresponding to the classification pattern, which is the correct answer data, is superimposed, and the MRCP image shown in C93 mentioned above are output from the trained model 72 using the display device interface 60 as the output section, and are displayed on the display device 90.

Based on the above, in the information processing system 20 of the present embodiment, the processor 30 acquires an MRCP image in which a part of the lumen appears, and presumes the route of the lumen between the part of the lumen shown in the MRCP image and the papillary portion based on the endoscope image and the MRCP image. In this way, the route of the lumen can be presumed more accurately in the ERCP treatment.

Further, in the information processing system 20 of the present embodiment, the trained model 72 may be trained by the training data 74 based on MRCP images. In this way, the trained model 72 trained by machine learning using MRCP images and endoscope images can be constructed. This allows for more accurate presumption of the lumen route during the ERCP treatment.

Further, as mentioned above, the method of the present embodiment may also be realized as a medical system 10. FIG. 21 shows a configuration example of the medical system 10 of the present embodiment. The medical system 10 includes an endoscope 100, a treatment tool 400, and a control device 600. The control device 600 includes a drive control device 200 to which a connector 201 is connected, and a video control device 500 to which a connector 202 is connected. The endoscope 100 is detachably connected to the control device 600 using the connectors 201 and 202.

The medical system 10 is also referred to as an endoscope system. Further, if the endoscope 100 is made to be driven electrically, the medical system 10 can also be referred to as an electric endoscope system. Although FIG. 21 shows an example of the medical system 10 using an electrically driven endoscope 100, a part of the configurations of the endoscope 100 may be manually driven.

The control device 600 controls each section, such as the drive control device 200, the video control device 500, and the like. The drive control device 200 controls the electrical driving of the endoscope 100 via the connector 201. Although not shown in FIG. 21, an operation device for manually operating the electrical driving may be connected to the drive control device 200. The video control device 500 receives an image signal from an imaging section provided at the distal end section 130 of the endoscope 100 via the connector 202, generates a display image from the image signal, and displays it on the display device 90 (not shown in FIG. 21).

In FIG. 21, the drive control device 200 and the video control device 500 are shown as separate devices; however, they may be structured as a single device. In this case, the connectors 201 and 202 may be integrated into a single connector.

The endoscope 100 includes an insertion section 110. The insertion section 110 is a portion to be inserted into a lumen of a patient, and is configured in a soft elongated shape. An insertion opening 190 of the treatment tool 400 is provided at the base end side of the insertion section 110, and a treatment tool channel for allowing the treatment tool 400 to pass through from the insertion opening 190 to the opening of the distal end section 130 is provided inside the insertion section 110. The insertion opening 190 of the treatment tool 400 is also called a forceps opening; however, the treatment tool to be used is not limited to forceps.

The configuration example of the medical system 10 of the present embodiment is not limited to the above configuration, and may further include an overtube 710 and a balloon 720, as shown, for example, in FIG. 21. The overtube 710 is a tube with variable hardness that covers the insertion section 110 of the endoscope 100. When the endoscope 100 and the overtube 710 are inserted into the body, at least the bending section of the insertion section 110 is exposed from the distal end of the overtube 710. The bending section refers to a section structured to be bent at an angle corresponding to the bending operation in the vicinity of the distal end of the insertion section 110. The base end of the overtube 710 is present outside the body. The base end side of the insertion section 110 is exposed from the base end of the overtube 710. The balloon 720 is provided near the distal end on the outer side of the overtube 710. For example, the operator performs an operation to inflate the balloon 720 provided near the distal end of the overtube 710, and fixes the distal end of the overtube 710 to the duodenum by the balloon 720. In this way, the position of the distal end of the overtube 710 can be fixed. The operator then performs, for example, an operation to harden the overtube 710. In this way, the insertion section 110 is held, thereby fixing the insertion route of the insertion section 110. This allows the insertion route of the insertion section 110 to be held. Since the method of hardening the overtube 710 is publicly known, the explanation thereof is omitted.

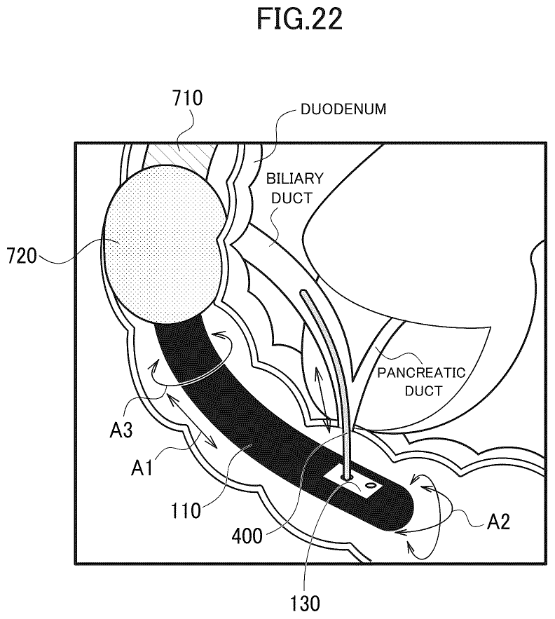

FIG. 22 shows the vicinity of the distal end of an endoscope positioned by the overtube 710 and the balloon 720. As shown in FIG. 22, the balloon 720 is fixed at a position slightly apart from the papillary portion to the pyloric side of the stomach. More specifically, the balloon 720 is positioned closer to the base end of the insertion section 110 than the base end of the bending section of the insertion section 110. By combining such a balloon 720 with the overtube 710 having a variable hardness, the bending section exposed to the papillary portion side from the balloon 720 and the distal end section 130 can be freely operated without being fixed, and the electrical driving from the base end side can be efficiently transmitted to the distal end section 130 of the endoscope.

The electrically-driven endoscopic operation is the forward and backward movement shown in A1, a bending movement shown in A2, or a rolling rotation shown in A3. The forward movement is a shift toward the distal end side along the axial direction of the insertion section 110, and the backward movement is a shift toward the base end side along the axial direction of the insertion section 110. The bending movement is a movement by which the angle of the distal end section 130 is changed due to the bending of the bending section. The bending movement includes bending movements in two directions orthogonal to each other, which can be controlled independently. One of the two directions orthogonal to each other is referred to as the vertical direction and the other is referred to as the horizontal direction. The rolling rotation is a rotation about an axis of the insertion section 110.

FIG. 22 shows an example in which the balloon 720 is attached to the distal end of the overtube 710 and the endoscope protrudes from the distal end of the overtube 710. However, it is sufficient that the overtube 710 and the balloon 720 are configured so that a portion of the bending section beyond the base end can freely move. For example, it may also be arranged such that a soft tube with a fixed hardness extends beyond the overtube with a variable hardness, and the balloon 720 is attached to the boundary thereof. In this case, although a part of the base end side of the bending section is covered with the soft tube, its movement is not hindered.

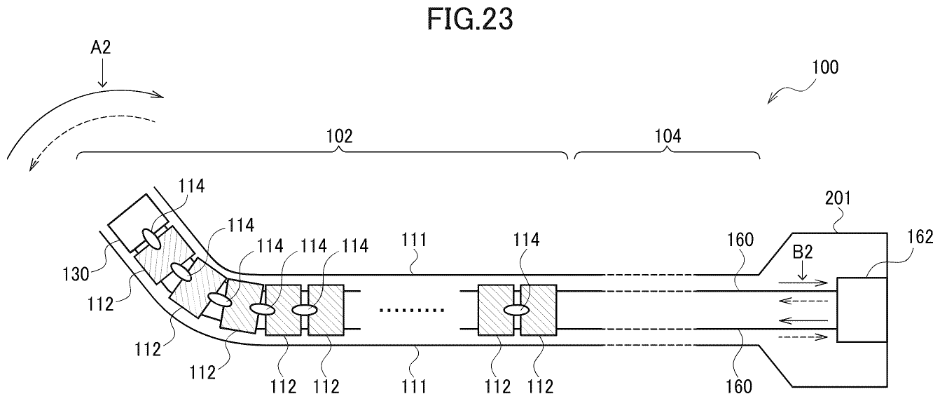

FIG. 23 is a schematic view of an endoscope 100 including a bending section 102 and a driving mechanism thereof. An endoscope 100 includes a bending section 102, a soft section 104, and a connector 201. The bending section 102 and the soft section 104 are covered with an outer sheath 111. The bending section 102 includes a plurality of bending pieces 112 and a distal end section 130 connected to the distal end of the bending pieces 112. The plurality of bending pieces 112 and the distal end section 130 are connected one another in series from the base end side to the distal end side by rotatable connecting sections 114, thereby forming a multi-joint structure. The connector 201 is provided with a coupling mechanism 162 on the endoscope side connected to a coupling mechanism on the drive control device 200 side. By attaching the connector 201 to the drive control device 200, it is possible to electrically drive the bending movement. Also, a bending wire 160 is provided in the outer sheath 111. One end of the bending wire 160 is connected to the distal end section 130. The bending wire 160 passes through the soft section 104 by penetrating through the plurality of bending pieces 112, turns back in the coupling mechanism 162, passes through the soft section 104 again, penetrates through the plurality of bending pieces 112. The other end of the bending wire 160 is connected to the distal end section 130. The driving force from the wire drive section of the drive control device 200 is transmitted to the bending wire 160 via the coupling mechanism 162 as the pulling force of the bending wire 160.

As shown by the solid line arrow B2 in FIG. 23, when the upper wire in the figure is pulled, the lower wire is pushed, whereby the multiple joints of the bending pieces 112 are bent upward in the figure. As a result, as indicated by the solid line arrow A2, the bending section 102 is curved upward in the figure. When the lower wire in the figure is pulled as indicated by the dotted arrow B2, similarly, the bending section 102 is curved downward in the figure as indicated by the dotted arrow A2. As described with reference to FIG. 22, the bending section 102 can be curved independently in two orthogonal directions. Although FIG. 23 shows a bending mechanism for one direction, two sets of bending wires are actually provided, and each bending wire can be curved independently in two directions by being pulled independently by the coupling mechanism 162.

Note that the mechanism for the electrically-driven bending is not limited to that described above. For example, a motor unit may be provided instead of the coupling mechanism 162. Specifically, it may be arranged such that the drive control device 200 transmits a control signal to the motor unit via the connector 201, and the motor unit drives the bending movement by pulling or relaxing the bending wire 160 based on the control signal.

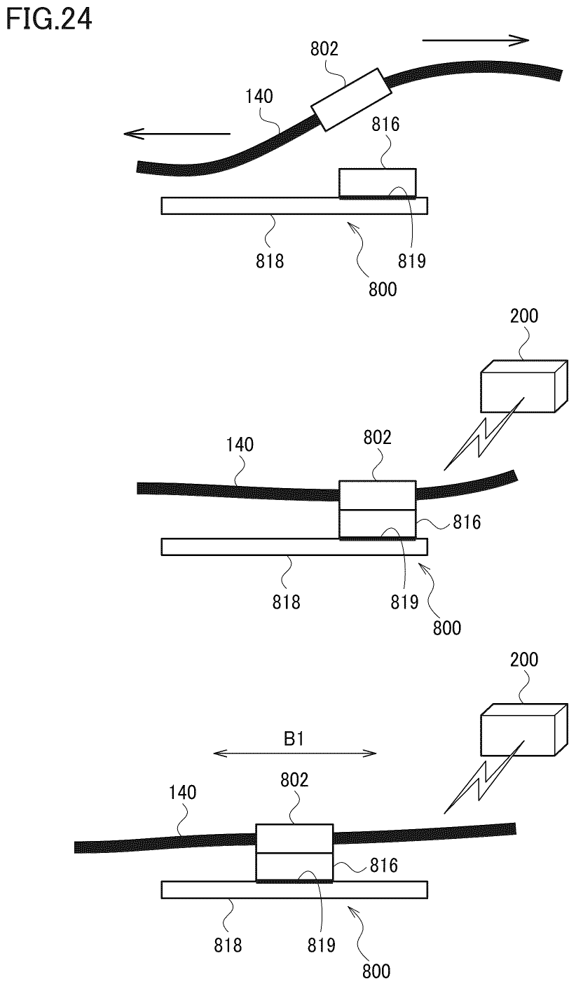

FIG. 24 shows a detailed configuration example of a forward/backward drive device 800. The forward/backward drive device 800 includes a motor unit 816, a base 818, and a slider 819. As shown in the upper and middle figures, an extracorporeal soft section 140 of the endoscope 100 is provided with an attachment 802 detachable from the motor unit 816. As shown in the middle figure, attaching the attachment 802 to the motor unit 816 enables electrical driving of forward/backward movement. As shown in the lower figure, the slider 819 supports the motor unit 816 while enabling the motor unit 816 to move linearly with respect to the base 818. The slider 819 is fixed to an operating table. As shown in B1, the drive control device 200 transmits a forward or backward control signal to the motor unit 816 by wireless communication, and the motor unit 816 and the attachment 802 move linearly on the slider 819 based on the control signal. As a result, the forward and backward movement of the endoscope 100 shown in A1 in FIG. 22 is achieved. Note that the drive control device 200 and the motor unit 816 may be connected by wired connection.

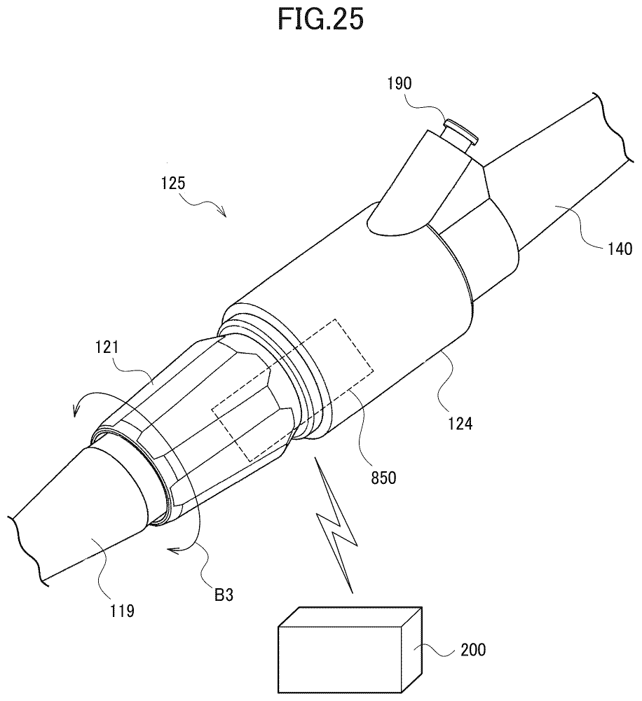

FIG. 25 is a perspective view of the connecting section 125 including a rolling drive device 850. The connecting section 125 includes a connecting section main body 124 and the rolling drive device 850. The insertion opening 190 of the treatment tool 400 is provided in the connecting section main body 124 and is connected to the treatment tool channel inside the connecting section main body 124. The connecting section main body 124 has a cylindrical shape, and a cylindrical member coaxial with the cylinder is rotatably provided inside the connecting section main body 124. The base end section of the intracorporeal soft section 119 is fixed to the outside of the cylindrical member, and the base end section serves as a rolling operation section 121. As a result, the intracorporeal soft section 119 and the cylindrical member can rotate with respect to the connecting section main body 124 about the axial direction of the intracorporeal soft section 119. The rolling drive device 850 is a motor unit provided inside the connecting section main body 124. As shown in B3, the drive control device 200 transmits a rolling rotation control signal to the rolling drive device 850 by wireless communication, and the rolling drive device 850 rotates the base end section of the intracorporeal soft section 119 with respect to the connecting section main body 124 based on the control signal, thereby causing rolling rotation of the intracorporeal soft section 119. As a result, the rolling rotation of the endoscope 100 shown in A3 in FIG. 22 is achieved. The rolling drive device 850 may include a clutch mechanism, and the rolling rotation may be switched between non-electrical driving and electrical driving by the clutch mechanism. Further, the drive control device 200 and the rolling drive device 850 may be connected by wired connection via a signal line passing through an internal route 101.

As mentioned above, since ERCP is a highly difficult task for operators, manually operating each section of the endoscope 100 is burdensome for the operators. By providing such an electrically driven medical system 10, the endoscopic operation, which is at least one of the forward and backward movements of the insertion section 110, the bending angle of the bending section of the insertion section 110, and the rolling rotation of the insertion section, is electrically driven, thereby reducing the burden on the operator.

At least some of the drive mechanisms shown in FIG. 23 to FIG. 25 may be applied to the treatment tool 400. For example, the treatment tool 400 shown in FIG. 21 may be connected to the control device 600, and the forward and backward movements, the bending movements, and the rolling driving movements of the treatment tool 400 may be electrically driven.

Further, as mentioned above in FIG. 19, when the captured endoscope image is corrected, the control of at least a part of the electrical driving may be changed according to the correction information. For example, when correction is made to rotate the endoscope image 180 degrees, for example, when the operator transmits a signal to drive the upper wire so that the distal end of the treatment tool 400 is curved via an operation section (not shown), the control device 600 performs the control to drive the lower wire based on the signal. In this way, the distal end of the treatment tool 400 is curved in a direction opposite to the direction based on the operation by the operator; therefore, the bending direction of the treatment tool 400 can be controlled to correspond to the corrected endoscope image. This method can be extended to other drive mechanisms of the endoscope 100.

As described above, in the information processing system 20 of the present embodiment, the endoscope 100 is an endoscope in which the endoscopic movement, which is at least one of the forward and backward movements of the insertion section 110, the bending angle of the bending section 102 of the insertion section 110, and the rolling rotation of the insertion section 110, is electrically driven. The processor 30 also controls the electrically-driven endoscopic operation based on control information according to the correction information. In this way, the operating burden on the operator can be reduced when the endoscope 100 is operated with respect to the corrected endoscope image.

Although the embodiments to which the present disclosure is applied and the modifications thereof have been described above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to form various disclosures. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term (processor) cited with a different term (processing section/control section) having a broader meaning or the same meaning at least once in the specification and the drawings can be replaced by the different term in any place in the specification and the drawings.

1. An information processing system comprising:
a processor including hardware, the processor being configured to:
acquire an endoscope image from an endoscope in which a papillary portion appears in the endoscope image;
correct the endoscope image based on correction information which is identified by an organ structure;
determine route information of a lumen based on the corrected endoscope image.

2. The information processing system of claim 1, wherein the processor is configured to:
acquire organ structural data before surgery; and
determine the correction information based on the structural data.

3. The information processing system of claim 1, wherein the processor is configured to correct the endoscope image such that the endoscope image has a given positional relationship between an imaging section of the endoscope and the papillary portion.

4. The information processing system of claim 1, further comprising a memory that stores a trained model trained by training data, which is a data set including a training endoscope image as input data and the route information of the lumen as correct answer data,
wherein the processor is configured to determine the route information of the lumen by inputting the corrected endoscope image to the trained model.

5. The information processing system of claim 4, wherein the trained model is trained by training data based on the training endoscope image captured with an imaging section of the endoscope and the papillary portion in a given positional relationship.

6. The information processing system of claim 4, wherein the trained model is trained by training data based on a classification pattern of the papillary portion.

7. The information processing system of claim 4, wherein the trained model is trained by training data based on an MRCP image.

8. The information processing system of claim 4, wherein the trained model is trained by training data based on an ultrasound endoscope image.

9. The information processing system of claim 1, wherein the processor is configured to:
generate a display image, in which a route guide image to provide guidance to a route of the lumen leading to the papillary portion is superimposed on the endoscope image that has not been corrected, based on a presumption result of the route information of the lumen, and
control a display device to display the display image.

10. The information processing system of claim 1, wherein the processor is configured to:
generate a display image, in which a route guide image to provide guidance to a route of the lumen leading to the papillary portion is superimposed on the corrected endoscope image, based on a presumption result of the route information of the lumen, and
control a display device to display the display image.

11. The information processing system of claim 9, wherein the processor is configured to correct the route guide image to correspond to correction to the endoscope image.

12. The information processing system of claim 1, wherein the endoscope comprises at least one electrically driven operation selected from a group consisting of an electrically driven forward and backward movement of an insertion section, an electrically driven bending angle of a bending section of the insertion section, and an electrically driven rolling rotation of the insertion section, and
the processor is configured to control the electrically-driven operation based on control information corresponding to the correction information.

13. The information processing system of claim 1, wherein the processor is configured to:
acquire an MRCP image in which a part of the lumen appears, and
determine a route of the lumen between the papillary portion and the part of the lumen shown in the MRCP image based on the endoscope image and the MRCP image.

14. The information processing system of claim 1, wherein the processor is configured to:
correct the endoscope image by a correction process including image rotation correction, and
determine the route information of the lumen based on the corrected endoscope image.

15. The information processing system of claim 1, wherein the processor is configured to:
correct the endoscope image by a correction process including image inclination correction, and
determine the route information of the lumen based on the corrected endoscope image.

16. A medical system comprising:
the information processing system of claim 1; and
the endoscope.

17. An information processing method, the method comprising:
acquiring an endoscope image from an endoscope in which a papillary portion appears in the endoscope image;
correcting the endoscope image based on correction information which is identified by an organ structure;
determining route information of a lumen based on the corrected endoscope image.

18. The information processing method of claim 17, further comprising acquiring organ structural data before surgery and determining the correction information based on the structural data.

19. The information processing method of claim 17, wherein the correcting is correcting the endoscope image such that the endoscope image has a given positional relationship between an imaging section of the endoscope and the papillary portion.

20. The information processing method according to claim 17, wherein
the determining is determining the route information of the lumen by inputting the corrected endoscope image to a trained model that is stored in a memory and trained by a training data which is a data set including a training endoscope image as input data and the route information of the lumen as correct answer data.
